(19) United States
(12) Patent Application Publication
(10) Pub. No.: US 2009/0088449 A1
(43) Pub. Date: Apr. 2, 2009

(54) 4-ACYLAMINOPYRIDINE DERIVATIVE MEDIATED NEUROGENESIS

(75) Inventor: Carrolee Barlow, San Diego, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MITSUBISHI TANABE PHARMA CORPORATION, Osaka-shi (JP)

(21) Appl. No.: 12/278,473

(22) PCT Filed: Feb. 6, 2007
(86) PCT No.: PCT/US2007/003326

§ 371 Date: Nov. 10, 2008

Related U.S. Application Data
(60) Provisional application No. 60/771,090, filed on Feb. 7, 2006.

(51) Int. Cl. A61K 31/4355; A61P 25/00; C12N 5/00

(52) U.S. Cl. 514/291; 435/375

(57) ABSTRACT

The instant disclosure describes methods for treating diseases and conditions of the central and peripheral nervous system by stimulating or increasing neurogenesis. The invention includes methods based on use of a 4-acylaminopyridine derivative to stimulate or activate the formation of new nerve cells.

### CROSS REFERENCE TO RELATED APPLICATION

The present application claims the benefit of U.S. provisional application No. 60/771,090, filed Feb. 7, 2006, which is incorporated by reference herein in its entirety.

### FIELD OF THE INVENTION

The instant invention relates to methods for treating diseases and conditions of the central and peripheral nervous system by stimulating or increasing neurogenesis via a 4-acylaminopyridine derivative. The invention includes methods based on the application of a 4-acylaminopyridine derivative to stimulate or activate the formation of new nerve cells.

### BACKGROUND OF THE INVENTION

Neurogenesis is a vital process in the brains of animals and humans, whereby new nerve cells are continuously generated throughout the life span of the organism. The newly born cells are able to differentiate into functional cells of the central nervous system and integrate into existing neural circuits in the brain. Neurogenesis is known to persist throughout adulthood in two regions of the mammalian brain: the subventricular zone (SVZ) of the lateral ventricles and the dentate gyrus of the hippocampus. In these regions, multipotent neural progenitor cells (NPCs) continue to divide and give rise to new functional neurons and glial cells (for review Gage 2000). It has been shown that a variety of factors can stimulate adult hippocampal neurogenesis, e.g., adrenalectomy, voluntary exercise, enriched environment, hippocampus dependent learning and anti-depressants (Yehuda 1989, van Praag 1999, Brown J 2003, Gould 1999, Malberg 2000, Santarelli 2003). Other factors, such as adrenal hormones, stress, age and drugs of abuse negatively influence neurogenesis (Cameron 1994, McEwen 1999, Kuhn 1996, Eisch 2004).

U.S. Pat. No. 5,397,785 describes a number of 4-acylaminopyridine derivatives and compositions comprising them as well as their use in the treatment of senile dementia and Alzheimer's Disease. U.S. Pat. No. 6,884,805 describes polymorph crystals of a 4-acylaminopyridine derivative and their use in activating a malfunctioned cholinergic neuron that is associated with memory loss disturbances. Neither of these patents relate to a use of a 4-acylaminopyridine derivative in relation to neurogenesis.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### BRIEF SUMMARY OF THE INVENTION

Disclosed herein are methods for the prophylaxis and treatment of diseases, conditions and injuries of the central and peripheral nervous systems by stimulating or increasing neurogenesis. Aspects of the invention include increasing neurogenesis in cases of a disease, disorder, or condition of the nervous system. Embodiments of the invention include methods of treating a neurodegenerative disorder, neurological trauma including brain or central nervous system trauma and/or recovery therefrom, depression, anxiety, psychosis, learning and memory disorders, and ischemia of the central and/or peripheral nervous systems.

In one aspect, the invention includes methods of stimulating or increasing neurogenesis. The neurogenesis may be at the level of a cell or tissue. The cell or tissue may be present in an animal subject or a human being, or alternatively be in an in vitro or ex vivo setting. In some embodiments, neurogenesis is stimulated or increased in a neural cell or tissue, such as that of the central or peripheral nervous system of an animal or human being. In cases of an animal or human, the methods may be practiced in connection with one or more disease, disorder, or condition of the nervous system as present in the animal or human subject. Thus, embodiments of the invention include methods of treating a disease, disorder, or condition by administering a neurogenic agent as described herein.

In another aspect, the invention includes methods of using chemical entities as neurogenic agents to increase neurogenesis. In some embodiments, a chemical entity is a 4-acylaminopyridine derivative, such as those described in U.S. Pat. No. 5,397,785, which is expressly incorporated herein by reference as if fully set forth. In one non-limiting embodiment, the derivative is 2-(2-oxypyrrolidin-1-yl)-N-(2,3-dimethyl-5,6,7,8-tetrahydrofuro(2,3-b)quinolin-4-yl)acetoamide. In other embodiments, the derivative is in a polymorph crystal form as described in U.S. Pat. No. 6,884,805, which is expressly incorporated herein by reference as if fully set forth. Of course the invention includes the use of more than one derivative. In further embodiments, the invention provides for the use of one or more derivatives in combination with another neurogenic agent.

In another aspect, the methods include identifying a patient suffering from one or more disease, disorders, or conditions, or a symptom thereof, and administering to the patient at least one neurogenic agent as described herein. As one non-limiting example, the agent is a 4-acylaminopyridine derivative, like 2-(2-oxypyrrolidin-1-yl)-N-(2,3-dimethyl-5,6,7,8-tetrahydrofuro(2,3-b)quinolin-4-yl)acetoamide as a non-limiting example. In some embodiments, the invention provides a method including identification of a subject as in need of an increase in neurogenesis, and administering to the subject one or more neurogenic agent as described herein. In other embodiments, the subject is a patient, such as a human patient.

The invention further provides a method including administering one or more neurogenic agent to a subject exhibiting the effects of insufficient amounts of, or inadequate levels of, neurogenesis. In some embodiments, the subject may be one that has been subjected to an agent that decreases or inhibits neurogenesis. Non-limiting examples of an inhibitor of neurogenesis includes opioid receptor agonists, such as a mu receptor subtype agonist like morphine. In a related manner, the invention provides for administering one or more neurogenic agent to a subject or person that will be subjected to an agent that decreases or inhibits neurogenesis. In some embodiments, the subject or person may be one that is about to be administered morphine or other opioid receptor agonist, like another opiate, and so about to be subject to a decrease or inhibition of neurogenesis. Non-limiting examples include administering a neurogenic agent to a subject before, simultaneously with, or after the subject is administered morphine or other opiate in connection with a surgical procedure.

Also disclosed are methods for preparing a population of neural stem cells suitable for transplantation, comprising culturing a population of neural stem cells (NSCs) in vitro, and contacting the cultured neural stem cells with at least one neurogenic agent of the invention. In some embodiments, the stem cells are prepared and then transferred to a recipient host animal or human. Non-limiting examples of preparation include 1) contact with a neurogenic agent until the cells have undergone neurogenesis, such as that which is detectable by visual inspection or cell counting, or 2) contact with a neurogenic agent until the cells have been sufficiently stimulated or induced toward or into neurogenesis. The cells prepared in such a non-limiting manner may be transplanted to a subject, optionally with simultaneous, nearly simultaneous, or subsequent administration of a neurogenic agent to the subject. While the neural stem cells may be in the form of an in vitro culture or cell line, in other embodiments, the cells may be part of a tissue which is subsequently transplanted into a subject.

In yet another aspect, the invention includes methods of stimulating or increasing neurogenesis in a subject by administering a 4-acylaminopyridine derivative and one or more additional neurogenic agents. In some embodiments, the neurogenesis occurs in combination with the stimulation of angiogenesis which provides new cells with access to the circulatory system.

The details of additional embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 is a dose-response curve showing effect of the neurogenic agent MKC-231 on neuronal differentiation. Data is presented as the percentage of the neuronal positive control, with basal media values subtracted. EC₅₀ was observed at an MKC-231 concentration of 5.1 μM in test cells, compared to 4.7 μM in positive control cells.

FIG. 2 is a dose-response curve showing effect of the neurogenic agent MKC-231 on astrocyte differentiation. Data is presented as the percentage of the astrocyte positive control, with basal media values subtracted. EC₅₀ was undeterminable for MKC-231 (greater than concentrations tested), compared to 19.9 μM in positive control cells.

FIG. 3 is a dose-response curve measuring the toxicity/trophism effect of the neurogenic agent MKC-231 on a population of cultured neural stem cells. Data is presented as the percentage of the basal media cell count.

FIG. 4 is a dose-response curve showing enhancement of the effects of the agent MKC-231 on neuronal differentiation by combination with an AMPA agonist (AMPA). Data is presented as the percentage of the neuronal positive control, with basal media values subtracted. EC₅₀ was observed at an MKC-231 concentration of 0.99 μM in combination with AMPA, compared to 5.1 μM with MKC-231 alone.

FIG. 5 is a series of immunofluorescent microscopic images of monolayers of human neural stem cells (hNSC) after immunohistochemistry staining with the neuronal marker TUJ-1 (green), the astrocyte marker GFAP (red), and a nuclear cell marker (Hoechst 33342 in blue). The upper left image is a negative control (basal media), the upper middle image is a neuronal positive control (basal media plus a known promoter of neuronal differentiation), and the upper right image is an astrocyte positive control (basal media plus a known inducer of astrocyte differentiation). The lower left image shows the effect of 31.6 μM MKC-231 on hNSC differentiation and the lower right image shows the effect of 31.6 μM MKC-231 in combination with 0.3161 μM AMPA on neuronal differentiation.

FIG. 6 is the average does response curve of multiple experiments (N=6) showing enhancement of the effects of the agent MKC-231 on neuronal differentiation by combination with affixed concentration of AMPA agonist (0.32 μM AMPA). The concentration of AMPA used does not promote neuronal differentiation alone (dashed gray line). Data is presented as the percentage of the neuronal positive control, with basal media values subtracted. EC₅₀ was observed at an MKC-231 concentration of 0.22 μM when in the presence of a fixed concentration AMPA (dashed black line), compared to 3.7 μM with MKC-231 alone (solid black line).

FIG. 7 is a dose-response curve showing inhibition of the effects of the agent MKC-231 on neuronal differentiation by combination with an AMPA antagonist (NBQX). Data is presented as the percentage of the neuronal positive control, with basal media values subtracted. EC₅₀ was observed at an MKC-231 concentration of >31.6 μM in combination with AMPA, compared to 5.1 μM with MKC-231 alone.

FIG. 8 is a bar graph depicting the change in hippocampal neurogenesis (increase in new neurons) compared to vehicle control (±SEM). The y-axis represents percent change compared to vehicle control. Daily administration of 1.0 and 4.0 mg/kg BCI-540 for 28 days resulted in a 22% and 20% increase in new neurons within the granule cell layer of the dentate gyrus, respectively.

FIG. 9 is a bar graph depicting the change in latency to eat in the novelty suppressed feeding assay (an animal model of depression) compared to vehicle control (±SEM). The y-axis represents percent change compared to vehicle control. Daily administration of 1.0 mg/kg BCI-540 and 10.0 mg/kg fluoxetine for 21 days resulted in a 35% and 38% decrease in latency to eat, respectively.

FIG. 10 is a bar graph depicting the mean percent time spent on the open arms of an elevated plus maze (an animal model of anxiety) compared to vehicle control (±SEM). Daily administration of 1.0 mg/kg BCI-540 for 21 days resulted in a 20% increase in the time spent on the open arms. A single administration of the classical anxiolytic diazepam resulted in a 12% increase in time spent on the open arms.

### DETAILED DESCRIPTION OF MODES OF PRACTICING THE INVENTION

“Neurogenesis” is defined herein as proliferation, differentiation, migration and/or survival of a neural cell in vivo or in vitro. In various embodiments, the neural cell is an adult, fetal, or embryonic neural stem cell or population of cells. The cells may be located in the central nervous system or elsewhere in an animal or human being. The cells may also be in a tissue, such as neural tissue. In some embodiments, the neural cell is an adult, fetal, or embryonic progenitor cell or population of cells, or a population of cells comprising a mixture of stem cells and progenitor cells. Neural cells include all brain stem cells, all brain progenitor cells, and all brain precursor cells. Neurogenesis includes neurogenesis as it occurs during normal development, as well as neural regeneration that occurs following disease, damage or therapeutic intervention, such as by the treatment described herein.

A “neurogenic agent” is defined as a chemical agent or reagent that can promote, stimulate, or otherwise increase the amount or degree or nature of neurogenesis in vivo or ex vivo or in vitro relative to the amount, degree, or nature of neurogenesis in the absence of the agent or reagent. In some embodiments, treatment with a neurogenic agent increases neurogenesis if it promotes neurogenesis by at least about 5%, at least about 10%, at least about 25%, at least about 50%, at least about 100%, at least about 500%, or more in comparison to the amount, degree, and/or nature of neurogenesis in the absence of the agent, under the conditions of the method used to detect or determine neurogenesis. As non-limiting examples, the agent may be a small organic molecule that is a 4-acylaminopyridine derivative.

The term “stem cell” (or neural stem cell (NSC)), as used herein, refers to an undifferentiated cell that is capable of self-renewal and differentiation into neurons, astrocytes, and/or oligodendrocytes.

The term “progenitor cell” (e.g., neural progenitor cell), as used herein, refers to a cell derived from a stem cell that is not itself a stem cell. Some progenitor cells can produce progeny that are capable of differentiating into more than one cell type.

The present invention includes methods of increasing neurogenesis by contacting cells with a 4-acylaminopyridine derivative as a neurogenic agent. The cells may be in vitro or in vivo, and include cells that are present in a tissue or organ of a subject animal or human being. The 4-acylaminopyridine derivative may be any that stimulates or increases neurogenesis. In one non-limiting example, the derivative is 2-(2-oxypyrrolidin-1-yl)-N-(2,3-dimethyl-5,6,7,8-tetrahydrofuro(2,3-b)quinolin-4-yl)acetoamide (also known as MKC-231, or coluracetam, and identified by CAS Registry number 135463-81-9). The cells are those capable of neurogenesis, such as to result, whether by direct differentiation or by proliferation and differentiation, in differentiated neuronal or glial cells. Representative, and non-limiting examples of other 4-acylaminopyridine derivative compounds for use in the present invention are provided in the Examples section below.

Without being bound by theory, and while some 4-acylaminopyridine derivatives have been contemplated in connection to inhibition of acetylcholinesterase (AChE) activity, the instant invention is not believed to be related to AChE inhibition because MKC-231 does not have such inhibitory activity. Similarly, the invention is believed to be unrelated to derivative binding at muscarinic or nicotinic receptors. It is believed, however, that the neurogenic action of MKC-231 may be through AMPA potentiation or sensitization. These beliefs are offered to improve the understanding of the invention and do not necessarily limit the invention.

In applications to an animal or human being, the invention relates to a method of bringing cells into contact with a neurogenic agent, or an effective amount of the agent, in a manner that results in an increase in neurogenesis in comparison to the absence of the agent. A non-limiting example is in the administration of the agent to the animal or human being. The neurogenic agent may be considered as exogenously supplied to a cell or tissue.

In some embodiments, the term “animal” or “animal subject” refers to a non-human mammal, such as a primate, canine, or feline. In other embodiments, the terms refer to an animal that is domesticated (e.g. livestock) or otherwise subject to human care and/or maintenance (e.g. zoo animals and other animals for exhibition). In other non-limiting examples, the terms refer to ruminants or carnivores, such as dogs, cats, birds, horses, cattle, sheep, goats, marine animals and mammals, penguins, deer, elk, and foxes.

The present invention also relates to methods of treating diseases, disorders, and conditions of the central and/or peripheral nervous systems (CNS and PNS, respectively) by administering one or more neurogenic agents. As used herein, “treating” includes prevention, amelioration, alleviation, and/or elimination of the disease, disorder, or condition being treated or one or more symptoms of the disease, disorder, or condition being treated, as well as improvement in the overall well being of a patient, as measured by objective and/or subjective criteria. In some embodiments, treating is used for reversing, attenuating, minimizing, suppressing, or halting undesirable or deleterious effects of, or effects from the progression of, a disease, disorder, or condition of the central and/or peripheral nervous systems. In other embodiments, the method of treating may be advantageously used in cases where additional neurogenesis would replace, replenish, or increase the numbers of cells lost due to injury or disease as non-limiting examples.

Non-limiting examples of symptoms that may be treated with the methods described herein include abnormal behavior, abnormal movement, hyperactivity, hallucinations, acute delusions, combativeness, hostility, negativism, withdrawal, seclusion, memory defects, sensory defects, cognitive defects, and tension. Non-limiting examples of abnormal behavior include irritability, poor impulse control, distractibility, and aggressiveness.

In some embodiments, the methods of the invention comprise using a 4-acylaminopyridine derivative as the neurogenic agent. The invention thus includes methods of contacting a cell with a 4-acylaminopyridine derivative, or administering such a derivative to a subject, to result in neurogenesis. Some embodiments comprise the use of one derivative, such as MKC-231, or a combination of two or more derivatives, such as MKC-231 and another derivative, as a neurogenic agent.

In some embodiments, the neurogenic agent(s) used in the methods described herein are substantially inactive with respect to other receptors, such as muscarinic receptors, nicotinic receptors, dopamine receptors, and opioid receptors as non-limiting examples.

In some embodiments, a 4-acylaminopyridine derivative is administered to an animal or human subject to result in neurogenesis. A 4-acylaminopyridine derivative may thus be used to treat a disease, disorder, or condition as described herein. In other embodiments, the 4-acylaminopyridine derivative may be used to increase neurogenesis in vitro.

Neurogenic agents for use in embodiments of the invention include MKC-231 as described above. It is represented by the following structure:

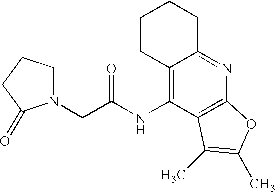

In some embodiments, the 4-acylaminopyridine derivative is one disclosed in U.S. Pat. No. 5,536,728 or a polymorph crystal form as disclosed in U.S. Pat. No. 6,884,805. Structures, biological activity data, methods for obtaining biological activity data, methods of synthesis, modes of administration and pharmaceutical formulations for such compounds are disclosed therein.

Methods for assessing the nature and/or degree of neurogenesis in vivo and in vitro, for detecting changes in the nature and/or degree of neurogenesis, for identifying neurogenesis modulating agents, for isolating and culturing neural stem cells, and for preparing neural stem cells for transplantation or other purposes are disclosed, for example, in U.S. Provisional Application No. 60/697,905, and U.S. Publication Nos. 2005/0009742 and 2005/0009847, 20050032702, 2005/0031538, 2005/0004046, 2004/0254152, 2004/0229291, and 2004/0185429, all of which are herein incorporated by reference in their entirety.

Neurogenesis includes the differentiation of neural cells along different potential lineages. In some embodiments of the invention, the differentiation of neural stem or progenitor cells is along a neuronal and/or glial cell lineage, optionally to the exclusion of differentiation along an astrocyte lineage.

Neurogenic agents described herein include pharmaceutically acceptable salts, derivatives, prodrugs, and metabolites of the agents. Methods for preparing and administering salts, derivatives, prodrugs, and metabolites of various agents are well known in the art.

Compounds described herein that contain a chiral center include all possible stereoisomers of the compound, including compositions comprising the racemic mixture of the two enantiomers, as well as compositions comprising each enantiomer individually, substantially free of the other enantiomer. Thus, for example, contemplated herein is a composition comprising the S enantiomer of a compound substantially free of the R enantiomer, or the R enantiomer substantially free of the S enantiomer. If the named compound comprises more than one chiral center, the scope of the present disclosure also includes compositions comprising mixtures of varying proportions between the diastereomers, as well as compositions comprising one or more diastereomers substantially free of one or more of the other diastereomers. By “substantially free” it is meant that the composition comprises less than 25%, 15%, 10%, 8%, 5%, 3%, or less than 1% of the minor enantiomer or diastereomer(s). Methods for synthesizing, isolating, preparing, and administering various stereoisomers are known in the art.

Methods described herein can be used to treat any disease or condition for which it is beneficial to promote or otherwise stimulate or increase neurogenesis. One focus of the methods described herein is to achieve a therapeutic result by increasing neurogenesis, as opposed to treating senile dementia, Alzheimer's Disease, or memory disturbances/memory loss. Thus, certain methods described herein can be used to treat any disease or condition susceptible to treatment by increasing neurogenesis. For example, in some embodiments, methods described herein are used to treat diseases or conditions that are not associated with significant dementia or memory issues, such as Parkinson's Disease, which is characterized by the degeneration of dopaminergic neurons. Thus, in one aspect, the present invention relates to the discovery of new therapeutic indications for a 4-acylaminopyridine derivative.

In some embodiments, the disease or condition being treated is associated with pain and/or addiction, but in contrast to known methods, the treatments of the invention are substantially mediated by increasing neurogenesis. For example, in some embodiments, methods described herein involve increasing neurogenesis ex vivo, such that a composition containing neural stem cells, neural progenitor cells, and/or differentiated neural cells can subsequently be administered to an individual to treat a disease or condition. In some embodiments, methods described herein allow treatment of diseases characterized by pain, addiction, and/or depression to be treated by directly replenishing, replacing, and/or supplementing neurons and/or glial cells. In further embodiments, methods described herein enhance the growth and/or survival of existing neural cells, and/or slow or reverse the loss of such cells in a neurodegenerative condition.

Examples of diseases and conditions treatable by the methods described herein include, but are not limited to, neurodegenerative disorders, such as Parkinson's disease, Parkinson's disorders, Huntington's disease (Huntington's Chorea), Lou Gehrig's disease, multiple sclerosis, Pick's disease, Parkinsonism dementia syndrome, progressive subcortical gliosis, progressive supranuclear palsy, thalmic degeneration syndrome, hereditary aphasia, amyotrophic lateral sclerosis, Shy-Drager syndrome, and Lewy body disease.

The invention also provides for the treatment of a nervous system disorder related to cellular degeneration, a psychiatric condition, cellular trauma and/or injury, or other neurologically related conditions. In practice, the invention may be applied to a subject or patient afflicted with, or diagnosed with, one or more central or peripheral nervous system disorders in any combination. Diagnosis may be performed by a skilled person in the applicable fields using known and routine methodologies which identify and/or distinguish these nervous system disorders from other conditions.

Non-limiting examples of nervous system disorders related to cellular degeneration include neurodegenerative disorders, neural stem cell disorders, neural progenitor cell disorders, degenerative diseases of the retina, and ischemic disorders. In some embodiments, an ischemic disorder comprises an insufficiency, or lack, of oxygen or angiogenesis, and non-limiting example include spinal ischemia, ischemic stroke, cerebral infarction, multi-infarct dementia. While these conditions may be present individually in a subject or patient, the invention also provides for the treatment of a subject or patient afflicted with, or diagnosed with, more than one of these conditions in any combination.

Non-limiting embodiments of nervous system disorders related to a psychiatric condition include neuropsychiatric disorders and affective disorders. As used herein, an affective disorder refers to a disorder of mood such as, but not limited to, depression, post-traumatic stress disorder (PTSD), hypomania, panic attacks, excessive elation, bipolar depression, bipolar disorder (manic-depression), and seasonal mood (or affective) disorder. Other non-limiting embodiments include schizophrenia and other psychoses, lissencephaly syndrome, anxiety syndromes, anxiety disorders, phobias, stress and related syndromes, cognitive function disorders, aggression, drug and alcohol abuse, obsessive compulsive behavior syndromes, borderline personality disorder, non-senile dementia, post-pain depression, post-partum depression, and cerebral palsy.

Examples of nervous system disorders related to cellular or tissue trauma and/or injury include, but are not limited to, neurological traumas and injuries, surgery related trauma and/or injury, retinal injury and trauma, injury related to epilepsy, spinal cord injury, brain injury, brain surgery, trauma related brain injury, trauma related to spinal cord injury, brain injury related to cancer treatment, spinal cord injury related to cancer treatment, brain injury related to infection, brain injury related to inflammation, spinal cord injury related to infection, spinal cord injury related to inflammation, brain injury related to environmental toxin, and spinal cord injury related to environmental toxin.

Non-limiting examples of nervous system disorders related to other neurologically related conditions include learning disorders, memory disorders, age-associated memory impairment (AAMI) or age-related memory loss, autism, attention deficit disorders, narcolepsy, sleep disorders, cognitive disorders, epilepsy, and temporal lobe epilepsy.

Additionally, the invention provides for the use of a neurogenic agent to treat a subject or patient for a condition due to the anti-neurogenic effects of an opiate or opioid based analgesic. In some embodiments of the invention, the administration of an opiate or opioid based analgesic, such as an opiate like morphine or other opioid receptor agonist, to a subject or patient results in a decrease in, or inhibition of, neurogenesis. The administration of a neurogenic agent of the invention in combination with an opiate or opioid based analgesic would reduce the anti-neurogenic effect. One non-limiting example is administration of a neurogenic agent of the invention in combination with an opioid receptor agonist after surgery (such as for the treating post-operative pain).

So the invention includes a method of treating post operative pain in a subject or patient by combining administration of an opiate or opioid based analgesic with a neurogenic agent of the invention. The analgesic may have been administered before, simultaneously with, or after a 4-acylaminopyridine derivative. In some cases, the analgesic or opioid receptor agonist is morphine or another opiate.

In other embodiments of the invention, the invention provides a method to treat or prevent decreases in or inhibition of neurogenesis in other cases involving use of an opioid receptor agonist. Non-limiting examples include cases involving an opioid receptor agonist, which decreases or inhibits neurogenesis, and drug addiction, drug rehabilitation, and/or prevention of relapse into addiction. In some embodiments, the opioid receptor agonist is morphine, opium or another opiate.

Compounds identified by methods of the invention can also be used to treat diseases of the of the peripheral nervous system (PNS), including but not limited to, PNS neuropathies (e.g., vascular neuropathies, diabetic neuropathies, amyloid neuropathies, and the like), neuralgias, neoplasms, myelin-related diseases, etc.

Other conditions that can be beneficially treated by increasing neurogenesis are known in the art (see e.g., U.S. Publication Nos. 20020106731, 2005/0009742 and 2005/0009847, 20050032702, 2005/0031538, 2005/0004046, 2004/0254152, 2004/0229291, and 2004/0185429, herein incorporated by reference in their entirety).

In some embodiments, the neurogenic agents used in the methods described herein comprise pharmaceutical compositions that include at least one pharmaceutically acceptable excipient. As used herein, the term “pharmaceutically acceptable excipient” includes any excipient known in the field as suitable for pharmaceutical application. Suitable pharmaceutical excipients and formulations are known in the art and are described, for example, in Remington's Pharmaceutical Sciences (19th ed.) (Genarro, ed. (1995) Mack Publishing Co., Easton, Pa.). Preferably, pharmaceutical carriers are chosen based upon the intended mode of administration of the neurogenic agent. The pharmaceutically acceptable carrier may include, for example, disintegrants, binders, lubricants, glidants, emollients, humectants, thickeners, silicones, flavoring agents, and water.

The neurogenic agent may be incorporated with excipients and administered in the form of ingestible tablets, buccal tablets, troches, capsules, elixirs, suspensions, syrups, wafers, or any other form known in the pharmaceutical arts. The pharmaceutical compositions of the invention may also be formulated in a sustained release form. Sustained release compositions, enteric coatings, and the like are known in the art. Alternatively, the compositions may be a quick release formulation.

In some embodiments, methods of treating according to the invention comprise the step of administering to a mammal a neurogenic agent, as defined herein, for a time and at a concentration sufficient to treat the condition targeted for treatment. Methods of the invention can be applied to individuals having, or who are likely to develop, disorders relating to neural degeneration, neural damage and/or neural demyelination. In some embodiments, methods described herein comprise a step of selecting a population or sub-population of patients, or selecting an individual patient, that is more amenable to treatment and/or less susceptible to side effects than other patients having the same disease or condition. For example, in some embodiments, a sub-population of patients is identified as being more amenable to neurogenesis with a neurogenic agent by taking a cell or tissue sample from prospective patients, isolating and culturing neural cells from the sample, and determining the effect of one or more neurogenic agents on the degree or nature of neurogenesis, thereby allowing selection of patients for which the therapeutic agent has a substantial effect on neurogenesis. Advantageously, the selection step(s) results in more effective treatment for the disease or condition that known methods using the same or similar compounds.

In other embodiments, the method of treatment comprises identifying, generating, and/or propagating neural cells ex vivo using one or more neurogenic agents, and transplanting the cells into a subject. In another embodiment, the method of treatment comprises the steps of contacting a neural stem cell of progenitor cell with one or more neurogenic agents to stimulate neurogenesis, and transplanting the cells into a patient in need of treatment. Also disclosed are methods for preparing a population of neural stem cells suitable for transplantation, comprising culturing a population of neural stem cells (NSCs) in vitro, and contacting the cultured neural stem cells with at least one neurogenic agent of the invention. The invention further includes methods of treating the diseases, disorders, and conditions described herein by transplanting such cells into a subject or patient.

Methods described herein may comprise administering to the subject an effective amount of a compound or pharmaceutical composition. In general, an effective amount of a compound according to the invention is an amount sufficient to stimulate or increase neurogenesis in the subject targeted for treatment when compared to the absence of the compound. An effective amount of a composition may vary based on a variety of factors, including but not limited to, the activity of the active compound(s), the physiological characteristics of the subject, the nature of the condition to be treated, and the route and/or method of administration. The methods of the invention typically involve the administration of an agent of the invention in a dosage range of 0.001 ng/kg/day to 500 ng/kg/day, preferably in a dosage range of 0.05 to 200 ng/kg/day. Advantageously, methods described herein allow treatment of indications with reductions in side effects, dosage levels, dosage frequency, treatment duration, tolerability, and/or other factors.

In some embodiments of the methods described herein, the use of neurogenic agents having selective activity may allow effective treatment with substantially fewer and/or less severe side effects compared to existing treatments. For example, neurogenic agents with selectivity within the CNS, can reduce side effects associated with activity at opioid receptors outside the intended target tissue/organ. Established methods of treating various conditions of the CNS and PNS with compounds having activity against opioid receptors have been known to cause side effects including, but not limited to, sweating, diarrhea, flushing, hypotension, bradycardia, bronchoconstriction, urinary bladder contraction, nausea, vomiting, parkinsonism, and increased mortality risk. In some embodiments, methods described herein allow treatment of certain conditions with doses that minimize these side effects.

Depending on the desired clinical result, the pharmaceutical compositions of the invention are administered by any means suitable for achieving a desired effect. Various delivery methods are known in the art and can be used to deliver a test agent to a subject or to NSCs or progenitor cells within a tissue of interest. The delivery method will depend on factors such as the tissue of interest, the nature of the compound (e.g., its stability and ability to cross the blood-brain barrier), and the duration of the experiment, among other factors. For example, an osmotic minipump can be implanted into a neurogenic region, such as the lateral ventricle. Alternatively, compounds can be administered by direct injection into the cerebrospinal fluid of the brain or spinal column, or into the eye. Compounds can also be administered into the periphery (such as by intravenous or subcutaneous injection, or oral delivery), and subsequently cross the blood-brain barrier.

In various embodiments, the pharmaceutical compositions of the invention are administered in a manner that allows them to contact the subventricular zone (SVZ) of the lateral ventricles and/or the dentate gyrus of the hippocampus. Examples of routes of administration include parenteral, e.g., intravenous, intradermal, subcutaneous, oral (e.g., inhalation), transdermal (topical), transmucosal, and rectal administration. Intranasal administration generally includes, but is not limited to, inhalation of aerosol suspensions for delivery of compositions to the nasal mucosa, trachea and bronchioli.

In some embodiments, the compounds of the invention are administered so as to either pass through or by-pass the blood-brain barrier. Methods for allowing factors to pass through the blood-brain barrier are known in the art, and include minimizing the size of the factor, providing hydrophobic factors which facilitate passage, and conjugating the neuro genic agent or other agent to a carrier molecule that has substantial permeability across the blood brain barrier. In some instances, the neurogenic agent can be administered by a surgical procedure implanting a catheter coupled to a pump device. The pump device can also be implanted or be extracorporally positioned. Administration of the neurogenic agent can be in intermittent pulses or as a continuous infusion. Devices for injection to discrete areas of the brain are known in the art. In a preferred embodiment, the neurogenic agent is administered locally to the ventricle of the brain, substantia nigra, striatum, locus ceruleous, nucleus basalis Meynert, pedunculopontine nucleus, cerebral cortex, and/or spinal cord by, e.g., injection. Methods, compositions, and devices for delivering therapeutics, including therapeutics for the treatment of diseases and conditions of the CNS and PNS, are known in the art.

In some embodiments, the delivery or targeting of neurogenic agents to a neurogenic region, such as the dentate gyrus or the subventricular zone, enhances efficacy and reduces side effects compared to known methods involving administration with the same or similar compounds.

In embodiments of the invention to treat subjects and patients, the methods include identifying a patient suffering from one or more disease, disorders, or conditions, or a symptom thereof, and administering to the subject or patient at least one neurogenic agent as described herein. The identification of a subject or patient as having one or more disease, disorder or condition, or a symptom thereof, may be made by a skilled practitioner using any appropriate means known in the field.

In further embodiments of the invention, the methods may be used to treat a cell, tissue, or subject which is exhibiting decreased neurogenesis or increased neurodegeneration. In some cases, the cell, tissue, or subject is, or has been, subjected to, or contacted with, an agent that decreases or inhibits neurogenesis. One non-limiting example is a human subject that has been administered morphine or other agent which decreases or inhibits neurogenesis. Non-limiting examples of other agents include opiates and opioid receptor agonists, such as mu receptor subtype agonists, that inhibit or decrease neurogenesis.

Thus in additional embodiments of the invention, the methods may be used to treat subjects having, or diagnosed with, depression or other withdrawal symptoms from morphine or other agents which decrease or inhibit neurogenesis. This is distinct from the treatment of subjects having, or diagnosed with, depression independent of an opiate, such as that of a psychiatric nature, as disclosed herein. In further embodiments, the methods may be used to treat a subject with one or more chemical addiction or dependency, such as with morphine or other opiates, where the addiction or dependency is ameliorated or alleviated by an increase in neurogenesis.

In embodiments comprising treatment of depression, the methods may further comprise use of one or more anti-depressant agents. Thus in the treatment of depression in a subject or patient, the method may comprise a neurogenic agent as described herein with one or more anti-depressant agents as known to the skilled person. Non-limiting examples of anti-depressant agents for use with a neurogenic agent of the invention include an SSRI, such as fluoxetine (Prozac®), citalopram, escitalopram, fluvoxamine, paroxetine (Paxil®), and sertraline (Zoloft®) as well as the active ingredients of known medications including Luvox® and Serozone®; selective norepinephrine reuptake inhibitors (SNRI) such as reboxetine (Edronax®) and atomoxetine (Strattera®); selective serotonin & norepinephrine reuptake inhibitor (SSNRI) such as venlafaxine (Effexor) and duloxetine (Cymbalta); and agents like baclofen, dehydroepiandrosterone (DHEA), and DHEA sulfate (DHEAS). In some embodiments, the use of a kappa opioid receptor subtype agonist and an SSRI, or baclofen, is used in the practice of the invention. The combination therapy may be advantageously used to improve the condition of the subject or patient. Non-limiting examples of combination therapy include the use of dosages which reduce side effects of an anti-depressant agent when used alone. For example, an anti-depressant agent like fluoxetine or paroxetine or sertraline may be administered at a reduced or limited dose, optionally also reduced in frequency of administration, in combination with a neurogenic agent of the invention. The reduced dose with the neurogenic agent mediates a sufficient anti-depressant effect so that the side effects of the anti-depressant agent alone are reduced or eliminated.

In embodiments for treating weight gain and/or to induce weight loss, a neurogenic agent of the invention may be used in combination with another agent for treating weight gain and/or inducing weight loss. Non-limiting examples of another agent for treating weight gain and/or inducing weight loss include various diet pills that are commercially available.

In other embodiments comprising combination therapy, methods of the invention comprise increasing neurogenesis in a subject or patient by administering a 4-acylaminopyridine derivative and one or more additional neurogenic agents or one or more neurogenesis modulating agent. So while the neurogenic agents of the invention can be administered as the sole active pharmaceutical agent, they can also be used in combination with one or more additional active agents, such as another neurogenic agent, optionally one that works by an alternative mechanism. When administered as a combination, the therapeutic agents can be formulated as separate compositions that are administered at the same time or sequentially at different times, or the therapeutic agents can be given as a single composition. The invention is not limited in the sequence of administration.

The additional neurogenic agent(s) may be an opioid or non-opioid (acts independently of an opioid receptor) agent. In some embodiments, the additional neurogenic agent is one that antagonizes one or more opioid receptors or is an inverse agonist of at least one opioid receptor. An opioid receptor antagonist or inverse agonist of the invention may be specific or selective (or alternatively non-specific or non-selective) for opioid receptor subtypes. So an antagonist may be non-specific or non-selective such that it antagonizes more than one of the three known opioid receptor subtypes, identified as OP₁, OP₂, and OP₃ (also know as delta, or δ, kappa, or κ, and mu, or μ, respectively). Thus an opioid that antagonizes any two, or all three, of these subtypes, or an inverse agonist that is specific or selective for any two or all three of these subtypes, may be used in the practice of the invention. Alternatively, an antagonist or inverse agonist may be specific or selective for one of the three subtypes, such as the kappa subtype as a non-limiting example.

In some embodiments, the additional neurogenic agent(s) used in the methods described herein has “selective” activity (such as in the case of an antagonist or inverse agonist) under certain conditions against one or more opioid receptor subtypes with respect to the degree and/or nature of activity against one or more other opioid receptor subtypes. For example, in some embodiments, the neurogenic agent has an antagonist effect against one or more subtypes, and a much weaker effect or substantially no effect against other subtypes. As another example, an additional neurogenic agent used in the methods described herein may act as an agonist at one or more opioid receptor subtypes and as antagonist at one or more other opioid receptor subtypes. In some embodiments, a neurogenic agent has activity against kappa opioid receptors, while having substantially lesser activity against one or both of the delta and mu receptor subtypes. In other embodiments, a neurogenic agent has activity against two opioid receptor subtypes, such as the kappa and delta subtypes. As non-limiting examples, the agents naloxone and naltrexone have nonselective antagonist activities against more than one opioid receptor subtypes. In certain embodiments, selective activity of one or more opioid antagonists results in enhanced efficacy, fewer side effects, lower effective dosages, less frequent dosing, or other desirable attributes.

An opioid receptor antagonist is an agent able to inhibit one or more characteristic responses of an opioid receptor or receptor subtype. As a non-limiting example, an antagonist may competitively or non-competitively bind to an opioid receptor, an agonist or partial agonist (or other ligand) of a receptor, and/or a downstream signaling molecule to inhibit a receptor's function.

An inverse agonist able to block or inhibit a constitutive activity of an opioid receptor may also be used. An inverse agonist may competitively or non-competitively bind to an opioid receptor and/or a downstream signaling molecule to inhibit a receptor's function. Non-limiting examples of inverse agonists for use in the practice of the invention include ICI-174864 (N,N-diallyl-Tyr-Aib-Aib-Phe-Leu), RTI-5989-1, RTI-5989-23, and RTI-5989-25 (see Zaki et al. J. Pharmacol. Exp. Therap. 298(3): 1015-1020, 2001).

In other embodiments, the additional neurogenic agent may be a modulator of a muscarinic receptor. Non-limiting examples of such agents include the muscarinic agonist milameline (CI-979) and xanomeline; the muscarinic agent alvameline (LU 25-109), 2,8-dimethyl-3-methylene-1-oxa-8-azaspiro[4,5]decane (YM-796) or YM-954, cevimeline (AF102B), sabcomeline (SB 202026), talsaclidine (WAL 2014 FU), CD-0102 ((5-(3-Ethyl-1,2,4-oxadiazol-5-yl)-1,4,5,6-tetrahydropyrimidine trifluoroacetic acid), 1-methyl-1,2,5,6-tetrahydropyridyl-1,2,5-thiadiazole derivative, such as tetra(ethyleneglycol)(4-methoxy-1,2, 5-thiadiazol-3-yl)[3-(1-methyl-1,2,5,6-tetrahydropyrid-3-yl)-1,2,5-thiadiazol-4-yl]ether, or a compound that is functionally or structurally related to a 1-methyl-1,2,5,6-tetrahydropyridyl-1,2,5-thiadiazole derivative, besipiridine, SR46559, L-689,660, S-9977-2, AF-102, or thiopilocarpine, an analog of clozapine or a diaryl[a,d]cycloheptene, such as an amino substituted form thereof, a benzimidazolidinone derivative, and a spiroazacyclic compound such as 1-oxa-3,8-diaza-spiro[4,5]decan-2-one, a tetrahydroquinoline analog; and a muscarinic ml receptor agonist selected from 55-LH-3B, 55-LH-25A, 55-LH-30B, 55-LH-4-1A, 40-LH-67,55-LH-15A, 55-LH-16B, 55-LH-11C, 55-LH-31A, 55-LH-46,55-LH-47, 55-LH4-3A.

In further embodiments, the additional agent may be one that increases the level of an endogenous muscarinic agonist, such as acetylcholine. Non-limiting examples of such additional agents include the acetylcholinesterase inhibitors Tacrine, Donepezil, Itoprid, Rivastigmine, and Galantamine.

In yet further embodiments, the additional neurogenic agent may be a modulator of an androgen receptor. Non-limiting examples include the androgen receptor agonists dehydroepiandrosterone (DHEA) and DHEA sulfate (DHEAS).

Of course a combination therapy may also be that of a neurogenic agent of the invention with a non-chemical based therapy. Non-limiting examples include the use of psychotherapy for the treatment of many conditions described herein, such as the psychiatric conditions, as well as behavior modification therapy such as that use in connection with a weight loss program.

Having now generally described the invention, the same will be more readily understood through reference to the following examples which are provided by way of illustration, and are not intended to be limiting of the present invention, unless specified.

### EXAMPLES

### Example 1

### Effect on Neuronal Differentiation of Human Neural Stem Cells

Human neural stem cells (hNSCs) were isolated and grown in monolayer culture, plated, treated with varying concentrations of MKC-231 (test compound), and stained with TUJ-1 antibody, as described in U.S. Provisional Application No. 60/697,905 (incorporated by reference). Mitogen-free test media with a positive control for neuronal differentiation was used along with basal media without growth factors as a negative control.

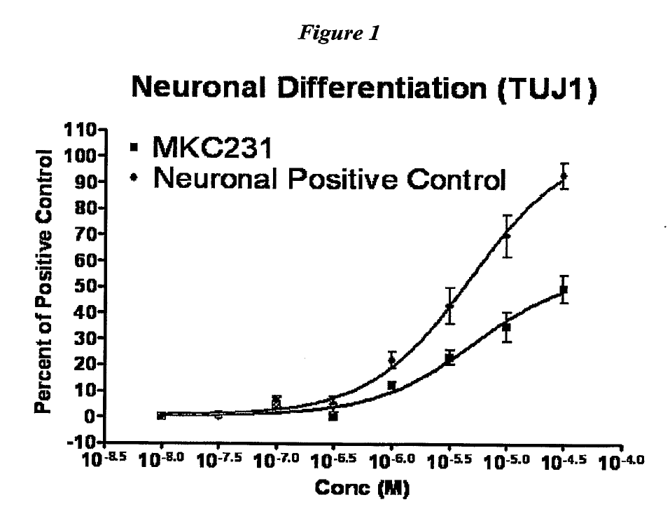

Results are shown in FIG. 1, which shows dose response curves of neuronal differentiation after background media values are subtracted. The dose response curve of the neuronal positive control is included as a reference. The data is presented as a percent of neuronal positive control. The data indicate that MKC-231 promoted neuronal differentiation above background levels.

### Example 2

### Effect on Astrocyte Differentiation of hNSCs

Experiments were carried out as described in Example 1, except the positive control for astrocyte differentiation contained mitogen-free test media with positive control for astrocyte differentiation, and cells were stained with GFAP antibody.

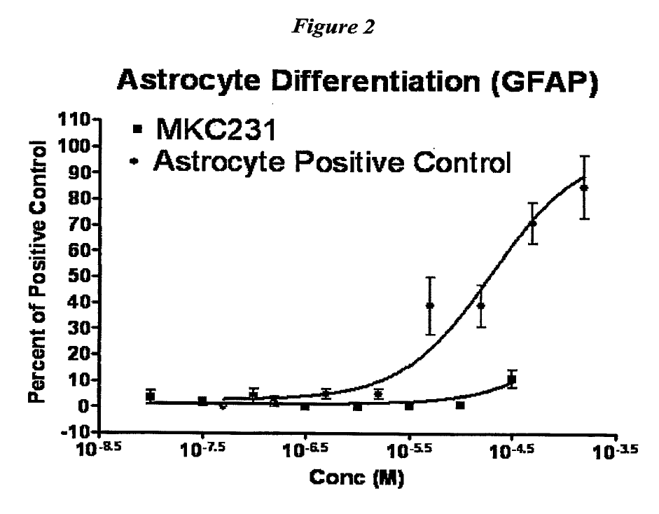

Results are shown in FIG. 2, which shows dose response curves of astrocyte differentiation after background media values are subtracted. MKC-231 showed no significant increase in astrocyte differentiation above basal media values.

### Example 3

### Toxic/Trophic Effect on Human Neural Stem Cells

Experiments were carried out as described in Example 1, except that the cells were stained with a nuclear dye (Hoechst 33342).

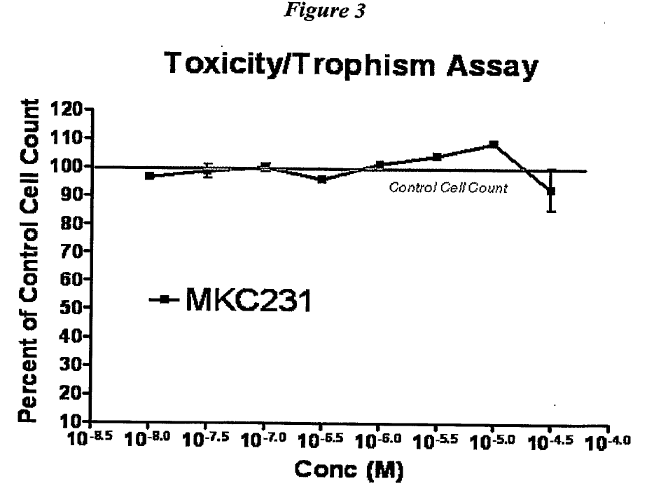

Results are shown in FIG. 3. The data therein is shown as a percentage of the basal media cell count. Concentrations that are toxic fall below 80% of the basal cell count. Trophic compounds show dose dependent increases in cell count. MKC-231 showed no toxicity at concentrations up to 31.6 μM.

### Example 4

### Effect of MKC-231 in Combination with an AMPA Agonist on Differentiation of Human Neural Stem Cells

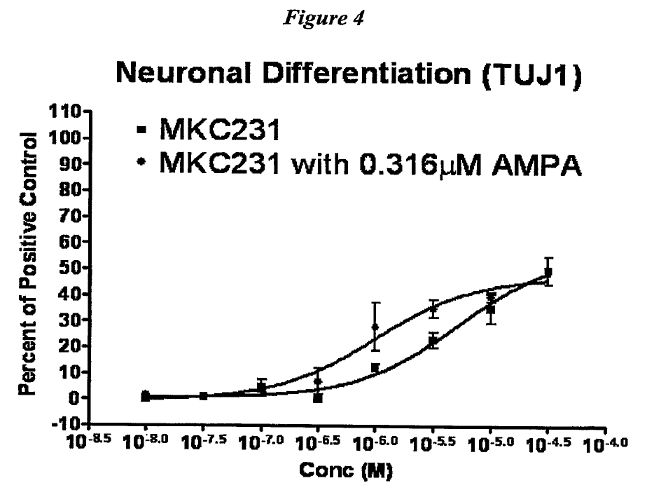

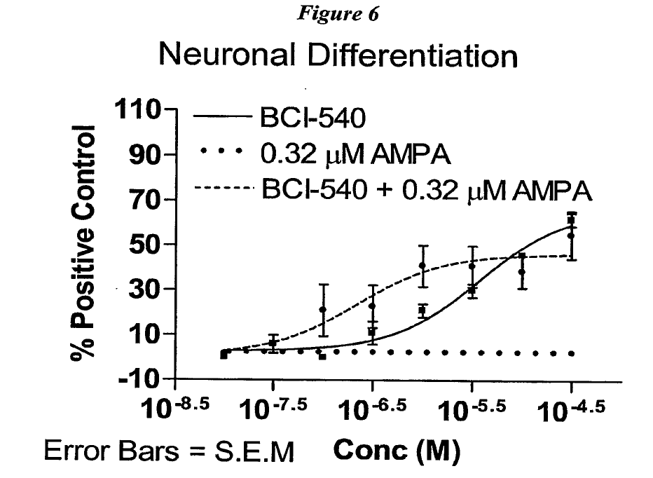

Experiments with various concentrations of MKC-231 alone or with 0.316 μM of an AMPA agonist (AMPA) were carried out generally as described in Example 1 for neuronal differentiation. The results are shown in FIG. 4, which shows dose response curves for neuronal differentiation after background media values are subtracted. 0.316 μM of the AMPA agonist AMPA enhances the stimulation of neuronal differentiation by the neurogenic agent MKC-231. These data demonstrate that the AMPA agonist acts as a potentiator or sensitizer of MKC-231 mediated neuronal differentiation. The data also indicate that MKC-231 apparently exerts some of its neurogenic effect as an AMPA potentiator or sensitizer. Also see FIG. 6 relating to BCI-540 in combination with AMPA and neuronal differentiation.

### Example 5

### Effect of MKC-231 in Combination with an AMPA Antagonist on Differentiation of Human Neural Stem Cells

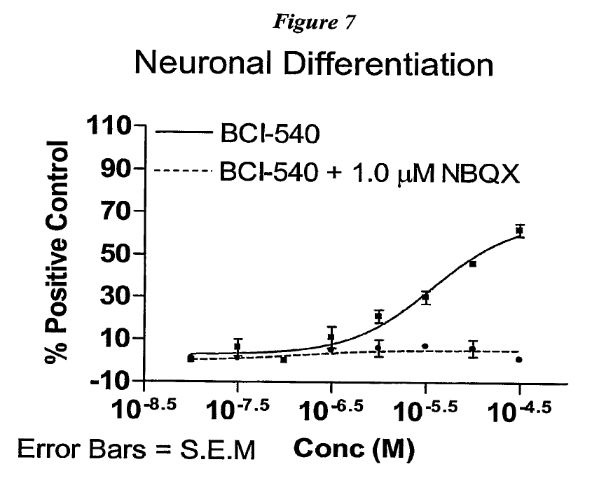

Experiments with various concentrations of MKC-231 alone or with 1.0 μM of an AMPA antagonist (NBQX) were carried out generally as described in Example 1 for neuronal differentiation. The results are shown in FIG. 7, which shows dose response curves for neuronal differentiation after background media values are subtracted. 1.0 μM of the AMPA antagonist NBQX inhibits the stimulation of neuronal differentiation by the neurogenic agent MKC-231. These data demonstrate that an AMPA antagonist acts as an inhibitor of MKC-231 mediated neuronal differentiation. The data also indicate that MKC-231 exerts some of its neurogenic effect as through the AMPA receptor activation.

### Example 6

### Effect on Hippocampal Neurogenesis in Rat

BCI-540 was administered to male F344 rats by oral gavage once daily for 28 days (1.0 and 4.0 mg/kg/day, p.o.). BrdU was administered once daily for 5 days (days 9 to 14, 100 mg/kg/day, i.p.). Animals were sacrificed on day 28. Brains were removed and processed for evaluation of neurogenesis.

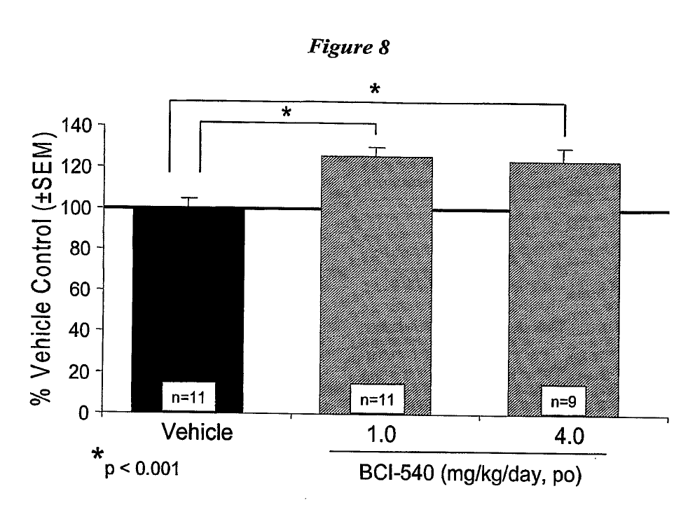

FIG. 8 shows the change in neurogenesis compared to vehicle control (±SEM). The y-axis represents percent change compared to vehicle control. The x-axis indicates treatment with vehicle set to 100% (black bar) and BCI-540 dosed at 1.0 and 4.0 mg/kg/day (black hatched bars). The black line indicates 100% of vehicle control. Daily administration of 1.0 and 4.0 mg/kg BCI-540 for 28 days resulted in a statistically significant increase in new neurons within the granule cell layer of the dentate gyrus.

### Example 7

### Effect in Novelty Suppressed Feeding in Rat

BCI-540 was administered to male F344 rats by oral gavage once daily for 21 days (1.0 mg/kg/day, p.o.). Fluoxetine was administered by oral gavage once daily for 21 days (10 mg/kg/day, p.o.). BrdU was administered once daily for 5 days (days 9 to 14, 100 mg/kg/day, i.p.). Animals were tested after 21 days of drug administration.

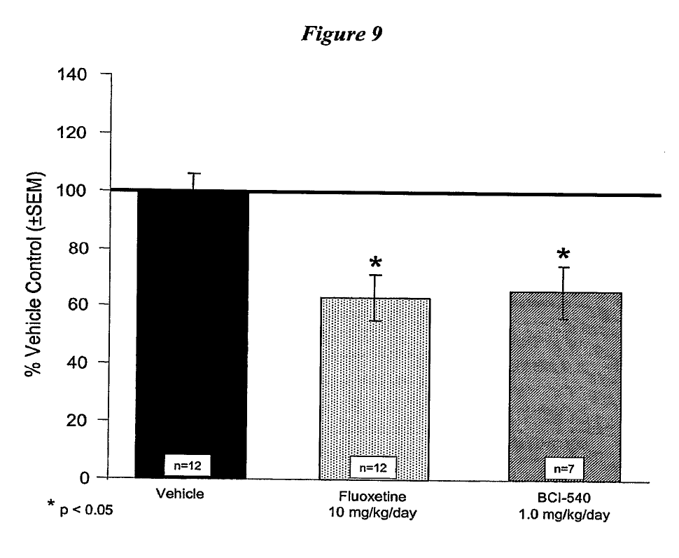

Shown in FIG. 9 is the change in latency to eat compared to vehicle control (±SEM). The y-axis represents percent change compared to vehicle control. The x-axis indicates treatment. Vehicle is set to 100%. The black line indicates 100% of vehicle control. 21-day administration of BCI-540 and fluoxetine resulted in a statistically significant decrease in latency to eat the food pellet, indicative of antidepressant activity.

### Example 8

### Toxic/Trophic Effect on Human Neural Stem Cells

BCI-540 was administered to male Sprague Dawley rats by oral gavage once daily for 21 days (1.0 mg/kg/day, p.o.). As a positive control, the anxiolytic diazepam (1.5 mg/kg, i.p.) was administered one time, 30 minutes prior to testing.

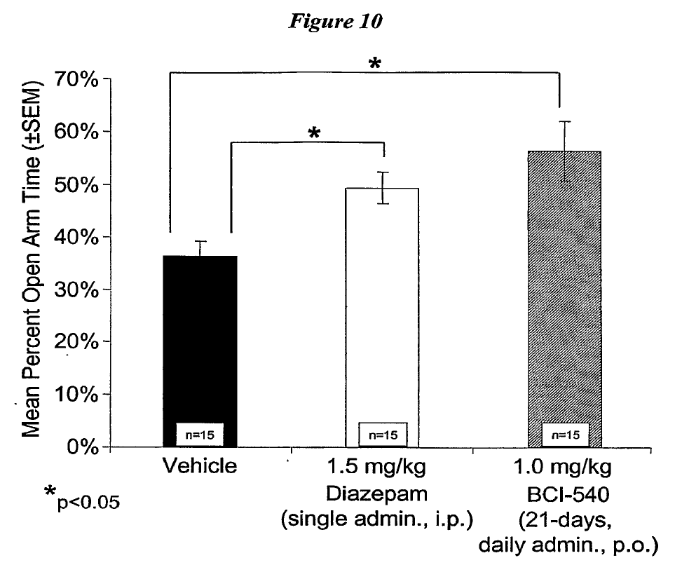

FIG. 10 shows the change in percent time spent on the open arms for the BCI-540 and diazepam treated groups (±SEM). The y-axis represents percent time on the open arm. The x-axis indicates treatment with vehicle (black bar), positive control diazepam (gray bar), and BCI-540 treated animals (black hatched bars) (n=15/group). Chronic administration of BCI-540 results in a significant increase in the time spent on the open arms which is indicative of anxiolytic activity. BCI-540 demonstrated anxiolytic efficacy comparable to acutely administered diazepam.

### Example 9

### Immunohistochemistry with Neuronal and Astrocyte Markers

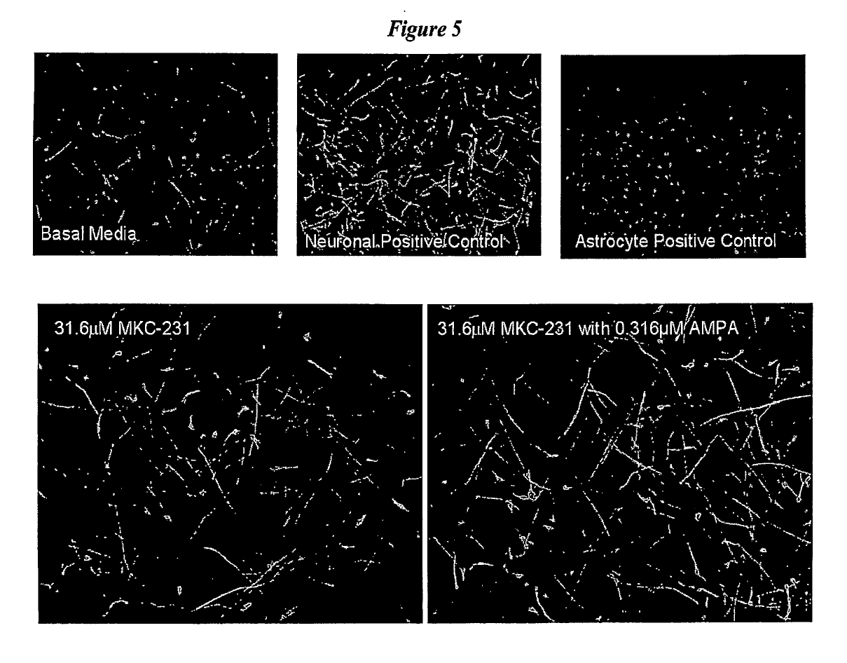

Immunohistochemistry was carried out as described in U.S. Provisional Application No. 60/697,905 (incorporated by reference) using TUJ-1 as a neuronal cell marker and GFAP as an astrocyte marker. The results are shown in FIG. 5, with control images included at the top for reference, and cells treated with 31.6 μM MKC-231 alone shown at the bottom left and the combination of 31.6 μM MKC-231 with 0.316 μM AMPA shown at the bottom right.

### Example 10

### Exemplary Neurogenic Agents

This example provides representative 4-acylaminopyridine derivatives for use in the various aspects and embodiments of the invention as disclosed above and below. A 4-acylaminopyridine derivative of the invention is represented by the following formula (1):

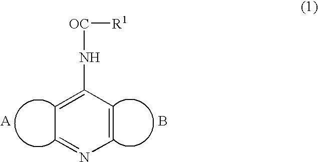

wherein R¹ represents a C₂-C₆ alkyl group or a group represented by the following formula (2):

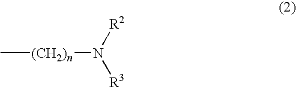

wherein each of R² and R³ independently represents a hydrogen atom, a C₁-C₆ alkyl group, a C₃-C₆ cycloalkyl group or a group represented by the following formula (3)

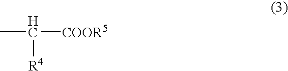

wherein each of R₄⁴ and R⁵ independently represents a hydrogen atom or a C₁-C₆ alkyl group, or R² and R³ together with the nitrogen atom to which both R² and R³ are attached represent

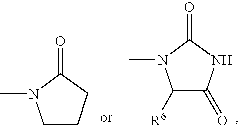

wherein R⁶ represents a hydrogen atom or C₁-C₆ alkyl group, and n represents 0 or an integer from 1 to 3; and

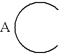

represents

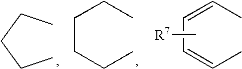

wherein R⁷ represents a hydrogen atom, a C₁-C₆ alkyl group or halogen atom,

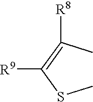

wherein each of R⁸ and R⁹ independently represents a hydrogen atom or a C₁-C₄ alkyl group,

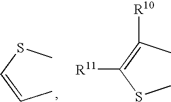

wherein each of R¹⁰ and R¹¹ independently represents a hydrogen atom or a C₁-C₄ alkyl group,

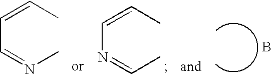

represents

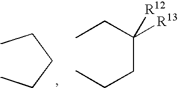

wherein each of R¹² and R¹³ independently represents a hydrogen atom or a C₁-C₄ alkyl group or R¹² and R¹³ may be combined together to form a C₂-C₆ alkylene group,

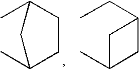

with the proviso that when R¹ is a C₂-C₆ alkyl group or a group represented by formula (2) wherein one of R² and R³ is a hydrogen atom or a C₁-C₆ alkyl group and the other of R² and R³ is a hydrogen atom or —CH₂COOR₅ wherein R⁵ is the same as defined above, or R² and R³ together with the nitrogen atom to which both R² and R³ are attached represent

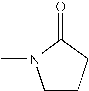

and n is 1 or 2,

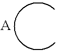

is not

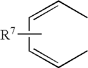

wherein R⁷ is the same as defined above, or is not

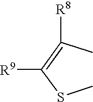

wherein R⁹ is the same as defined above; and

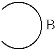

is not

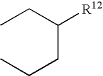

wherein R¹² represents a hydrogen atom or a C₁-C₄ alkyl group or is not

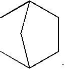

In formula (1), non-limiting examples of a C₂-C₆ alkyl group (or alkyl group having 2 to 6 carbon atoms) represented by R¹ include the following: ethyl group, n-propyl group, isopropyl group, n-butyl group, sec-butyl group, tert-butyl group, n-pentyl group and n-hexyl group may be mentioned. In some embodiments, a C₂-C₄ alkyl group is used in the methods and practice of the invention.

Non-limiting examples of a C₁-C₆ alkyl group represented by each of R² to R⁷ include the following: methyl group, ethyl group, n-propyl group, isopropyl group, n-butyl group, sec-butyl group, tert-butyl group, n-pentyl group and n-hexyl group. In some embodiments, a C₁-C₄ alkyl group is used in the methods and practice of the invention.

Non-limiting examples of a C₃-C₆ cycloalkyl group represented by each of R² and R³ include the following: cyclopropyl group, cyclobutyl group, cyclopentyl group and cyclohexyl group.

A halogen atom represented by R⁷ is selected from a fluorine atom, chlorine atom, bromine atom and iodine atom.

Non-limiting examples of a C₁-C₄ alkyl group represented by each of R⁸ to R¹³ include the following: methyl group, ethyl group, n-propyl group, isopropyl group, n-butyl group, sec-butyl group and tert-butyl group.

Among the compounds represented by formula (1), and in some embodiments of the invention, a compound wherein R¹ represents a C₂-C₆ alkyl group or a group represented by formula (2) wherein R² represents a hydrogen atom or a C₁-C₆ alkyl group, R³ represents a hydrogen atom, a C₁-C₆ alkyl group, a C₃-C₆ cycloalkyl group or a group represented by formula (3) wherein each of R⁴ and R⁵ independently represents a hydrogen atom or a C₁-C₆ alkyl group, or R² and R³ together with the nitrogen atom to which both R² and R³ are attached represent

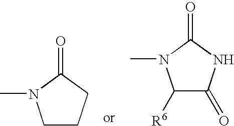

wherein R⁶ represents a hydrogen atom or C₁-C₆ alkyl group, and n represents 0 or an integer from 1 to 3; and

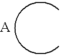

represents

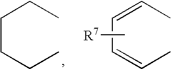

wherein R⁷ represents a hydrogen atom, a C₁-C₆ alkyl group or halogen atom,

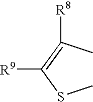

wherein each of R⁸ and R⁹ independently represents a hydrogen atom or a C₁-C₄ alkyl group, or

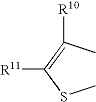

wherein each of R¹⁰ and R¹¹ independently represents a hydrogen atom or a C₁-C₄ alkyl group; and

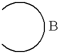

represents

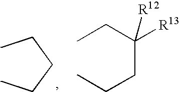

wherein each of R¹² and R¹³ independently represents a hydrogen atom or a C₁-C₄ alkyl group or R¹² and R¹³ may be combined together to form a C₂-C₆ alkylene group, or

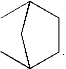

More preferred is a compound wherein

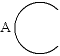

represents

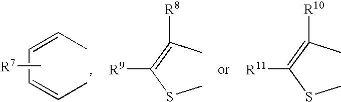

wherein R⁷ to R¹¹ are as defined above.

In addition to the above represented molecules, pharmaceutically acceptable acid-added salts of the molecules are also provided by the instant invention. In some embodiments, the acid-added salt of a compound represented by formula (1) is pharmaceutically and physiologically acceptable. As non-limiting examples, inorganic acid-added salts such as hydrochloride, hydrobromine, hydroiodide, sulfate and phosphate, and organic acid-added salts such as oxalate, maleate, fumarate, lactate, malate, citrate, tartrate, benzoate, methanesulfonate and camphorsulfonate are provided. The compound represented by formula (1) and the acid-added salt thereof can be present in the form of hydrate or solvate. The hydrate and solvate may also be used in the methods and practice of the present invention.

Methods for the preparation of the above are provided in U.S. Pat. No. 5,397,785, which is hereby incorporated by reference. Example 25 therein relates to the preparation of MKC-231.

### Example 11

### Exemplary Compositions and Dosages

In addition to the description of compositions described above, the present invention further provides additional compositions comprising a neurogenic agent herein. The composition may optionally include an additional neurogenic agent as described above. In some embodiments, the composition comprises a pharmaceutically effective amount of a 4-acylaminopyridine derivative represented by formula (1), as described above, or a pharmaceutically acceptable acid-added salt thereof, and a pharmaceutically acceptable adjuvant.

A neurogenic compound of the present invention may be used as a therapeutic agent or medicine by administrating it singly or in a mixture with a pharmaceutically acceptable carrier. Optionally, the compound may be formulated with one or more additional neurogenic agents as described herein. The composition may be determined as a matter of routine by a skilled person in the field based on the solubility and property of the compound to be used as the active ingredient, the administration route and dosage regimen. As a non-limiting example, the compound of the present invention may be orally administered in the form of granule, subtilized granule, powder, tablet, hard capsule, soft capsule, syrup, emulsion, suspension and solution. The compound of the present invention may be also administered intravenously, intramuscularly or subcutaneously by injection. The compound of the present invention may be prepared to an injectable powder and injected after dissolving or suspending in an appropriate solvent when used.

The compound may be used with an organic or inorganic, solid or liquid, carrier or diluent, which is suitable for oral, intestinal, parenteral or local administration. As non-limiting examples of a vehicle for a solid preparation, lactose, sucrose, starch, talc, cellulose, dextrin, kaolin and calcium carbonate are provided. A liquid preparation for oral administration, i.e. emulsion, syrup, suspension, solution, etc., may contain a diluent such as water, vegetable oil, etc as non-limiting examples. The liquid preparation may contain an auxiliary such as a humectant, suspending agent, sweetening agent, aromatic, coloring agent, preservative, etc in addition to an inert diluent. In some embodiments, the liquid preparation may be encapsulated in an absorbable wall substance such as gelatin. As a solvent or a suspending agent used for preparing a parenteral preparation such as an injection preparation, water, propylene glycol, polyethylene glycol, benzyl alcohol, ethyl oleate and lecithin may be used. The preparation of such compositions may be conducted out with use of standard techniques known to the skilled person.

The daily clinical dosage of the compound of the present invention in oral administration may be from about 1 to about 1000 mg, such as about 10 to about 100 mg for an adult. The skilled person may determine that it is desirable to increase or decrease the dose depending upon the age of patient, condition of disease, condition of patient, and whether or not another medicine or active agent is administered. The daily dose of the compound of the present invention may be administered in one, or in two or three portions with appropriate time intervals in between. Intermittent administration may also be used.

The daily dosage of the compound of the present invention in injection may be from about 0.1 to about 100 mg, such as from about 0.5 to about 50 mg for an adult.

Further, compounds of the invention are very low in toxicity and produce few side effects.

### Example 12

### Exemplary Crystal Forms

Pure or essentially pure crystal forms of a 4-acylaminopyridine derivative may also be used in the methods and practice of the invention. In some embodiments, a crystal form of N-(2,3-dimethyl-5,6,7,8-tetrahydrofuro[2,3-b]quinolin-4-yl)-2-(2-oxopyrrolidin-4-yl)acetamide (or MKC-231), as described in U.S. Pat. No. 6,884,805, is used in the methods and practice of the invention. The crystal may be form A or form B crystal. The patent also provides a description for the preparation, including preparation in physiologically acceptable solvents, of the crystal forms.

The form A crystal is characterized by one or more of the following: (i) a melting point obtained from a differential scanning calorimetry curve lower than about 220° C., particularly a melting point obtained from a differential scanning calorimetry curve of about 217.6° C., (ii) a peak at X-ray diffraction angle, 2θ, of 9.8° (±0.2°), (iii) the absence of a peak at X-ray diffraction angle, 2θ, of 7.3° (±0.2°), (iv) a water solubility of lower than about 0.5 mg/mL, particularly a water solubility of about 0.35 mg/mL, and (v) greater stability in storage than the form B crystal.

The form B crystal is characterized by one or more of the following: (i) a melting point obtained from a differential scanning calorimetry curve higher than about 220° C., particularly a melting point obtained from a differential scanning calorimetry curve of about 222.6° C., (ii) a peak at X-ray diffraction angle, 2θ, of 7.3° (±0.2°), (iii) the absence of a peak at X-ray diffraction angles, 2θ, of 9.8° (±0.2°), (iv) a water solubility of higher than about 0.5 mg/mL, particularly a water solubility of about 0.73 mg/mL, and (v) a lower stability in storage than the form A crystal.

The crystal form may be in the form of a bulk pharmaceutical comprising either the form A or form B crystal. Moreover, the invention includes a pharmaceutical composition comprising a pharmaceutically acceptable carrier and either the form A or the form B crystal.

In greater detail, the form A crystal is characterized by one or more of the following: (a) a melting point (extrapolated onset) obtained from a differential scanning calorimetry curve lower than about 220° C., such as in the range of about 213-220° C., or in the range of about 215-220° C., in the range of about 216-218° C., in the range of about 218° C., and in the range of about 217.6° C., (b) at least one peak in the X-ray diffraction spectrum at diffraction angle. 2θ, of 8.7°, 9.8°, 11.4°, 13.3°, 15.5°, 16.8°, and/or 17.6° (±0.2°, respectively), such as at least one peak in the X-ray diffraction spectrum at diffraction angle, 2θ, of 9.8° (±0.2°), (c) the absence of a peak (taking into account baseline noise and variations among instruments) at X-ray diffraction angle, 2θ, of 7.3°, 9.3°, 11.9°, and/or 14.8° (±0.2°, respectively), (d) a water solubility (at 25° C.) of lower than about 0.5 mg/mL, such as in the range of about 0.1-0.5 mg/mL, in the range of about 0.2-0.45 mg/mL, in the range of about 0.3-0.4 mg/mL, and in the range of about 0.35 mg/mL, and (e) a better stability at room temperature (about 25° C.) and during storage (over time) than the form B crystal.

The form B crystal is characterized by one or more of the following: (a) a melting point (extrapolated onset) obtained from a differential scanning calorimetry curve higher than about 220° C., such as in the range of about 220-225° C., in the range of about 221-224° C., in the range of about 222-223° C., in the range of about 223° C., and in the range of about 222.6° C., (b) at least one peak in the X-ray diffraction spectrum at diffraction angle, 2θ, of 7.3°, 9.3°, 11.9°, 13.5°, 14.8°, 15.9°, 17.5°, and/or 18.6° (±0.2°, respectively), such as at least one peak in the X-ray diffraction spectrum at diffraction angle, 2θ, of 7.3° (±0.2°), (c) the absence of a peak (taking into account baseline noise and variations among instruments) at X-ray diffraction angle, 2θ, of 8.7°, 9.8°, and/or 16.8° (±2′, respectively), (d) a water solubility (at about 25° C.) of higher than about 0.5 mg/mL, such as in the range of about 0.5-1 mg/mL, in the range of about 0.6-0.9 mg/mL, in the range of about 0.7-0.8 mg/mL, and in the range of about 0.73 mg/mL, and (e) a lower stability at room temperature (about 25° C.) and during storage (over time) than the form A crystal.

The invention further provides for stable, pure or essentially pure, crystal forms A and B of MKC-231. The crystal forms may be prepared with good reproducibility by using a physiologically compatible solvent, such as ethanol, water, or a mixture thereof. The term “essentially pure” indicates that either the form A crystal or the form B crystal contains less than about 10 wt. % of the other polymorph form, such as less than about 5 wt. % of the other polymorph form. Ideally, the aforementioned percentages refer to any other polymorph form to the extent there may exist polymorph forms other than form A and form B as described herein.

A crystal form may be used in a pharmaceutical composition comprising a pharmaceutically acceptable (e.g., physiologically acceptable or pharmacologically compatible) carrier for the treatment of the diseases, disorders, or conditions described herein. The crystal forms of the invention (and pharmaceutical compositions thereof), therefore, are useful in a method of treating a mammal, particularly a human, with a disease.

The route for administering form A or form B crystal of the invention is not particularly limited. The crystal form may be administered either orally or parenterally, optionally while remaining in crystal form. Alternatively, it is administered as a pharmaceutical composition containing the active ingredient and additives, which are pharmaceutically acceptable (e.g., pharmacologically compatible). The choice of carrier will be determined, in part, both by the particular composition and by the particular method used to administer the composition as described above.

Pharmaceutically acceptable additives can be employed. Non-limiting examples include vehicles, disintegrators, disintegrating aids, binders, lubricants, coating agents, pigments, diluents, bases, dissolving agents, dissolving aids, isotonizing agents, pH regulators, stabilizers, propellants, and adhesives. Non-limiting examples of preparations suitable for oral administration include tablets, capsules, powders, fine granules, granules, solutions and syrups. Non-limiting examples of preparations suitable for parenteral administration include injections, drops, ointments, creams, percutaneously absorbing agents, eye drops, ear drops, inhalants and suppositories. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. The form of the preparation of the pharmaceutical composition is not restricted to those recited herein.

Additional vehicles can be added to preparations suitable for oral administration. Suitable additives vehicles include glucose, lactose, D-mannitol, starch and crystalline cellulose; disintegrators or disintegrating aids such as carboxymethyl cellulose, starch and carboxymethyl cellulose calcium salt; binders such as hydroxypropyl cellulose, hydroxypropylmethyl cellulose, poly (vinyl pyrrolidone) and gelatin; lubricants such as magnesium stearate and talc; coating agents such as hydroxypropylmethyl cellulose, saccharose, polyethylene glycol and titanium oxide; and bases such as Vaseline, liquid paraffin, polyethylene glycol, gelatin, kaolin, glycerin, purified water and hard fat. Typical additives for preparations suitable for injections or eye drops include dissolving agents or dissolving aids that can constitute aqueous or dissolved-before-use injections such as distilled water for injection, physiological saline solutions and propylene glycol; isotonizing agents such as glucose, sodium chloride. D-mannitol and glycerin; pH regulators such as inorganic acids, organic acids, inorganic salts, and organic salts.

The dosage of the medicament of the invention may be appropriately increased or decreased depending on the disease, the purpose of the treatment (e.g., prevention or treatment), and the conditions of the patient such as age, weight, and symptoms. However, in general, the daily dosage for an adult patient by oral administration is about 0.05 to about 500 mg per day. In general, the aforesaid dosage can be administered one time, several times each day, or every several days.

The crystal forms may be further understood by reference to the following references: Chaki et al., Bioorganic & Medical Chemistry Letters, 5(14), 1489-1494 (1995); Chaki et al., Bioorganic & Medical Chemistry Letters, 5(14), 1495-1500 (1995); Bessho et al., Arznein.Forsh./Drug Res., 46(I), 369-373 (1996); Murai et al., J. Neuron. Transm. [GenSect], 98, 1-13 (1994); and Akaike et al., Jpn. J. Pharmacol., 76, 219-222 (1998).

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of treating a nervous system disorder related to cellular degeneration, a psychiatric condition, cellular trauma and/or injury, or another neurologically related condition in a subject or patient, said method comprising:
administering a 4-acylaminopyridine derivative to said subject or patient.

2. The method of claim 1, wherein said nervous system disorder related to cellular degeneration is selected from a neurodegenerative disorder, a neural stem cell disorder, a neural progenitor cell disorder, a degenerative disease of the retina, an ischemic disorder, and combinations thereof.

3. The method of claim 1, wherein said nervous system disorder related to a psychiatric condition is selected from a neuropsychiatric disorder, an affective disorder, depression, hypomania, panic attacks, anxiety, excessive elation, bipolar depression, bipolar disorder (manic-depression), seasonal mood (or affective) disorder, lissencephaly syndrome, anxiety syndromes, anxiety disorders, phobias, stress and related syndromes, cognitive function disorders, aggression, drug and alcohol abuse, obsessive compulsive behavior syndromes, borderline personality disorder, non-senile dementia, post-pain depression, post-partum depression, cerebral palsy, and combinations thereof.

4. The method of claim 3, wherein said nervous system disorder related to a psychiatric condition is selected from the group consisting of depression, bipolar depression, bipolar disorder (manic-depression), post-pain depression and postpartum depression.

5. The method of claim 1, wherein said nervous system disorder related to cellular trauma and/or injury is selected from neurological traumas and injuries, surgery related trauma and/or injury, retinal injury and trauma, injury related to epilepsy, spinal cord injury, brain injury, brain surgery, trauma related brain injury, trauma related to spinal cord injury, brain injury related to cancer treatment, spinal cord injury related to cancer treatment, brain injury related to infection, brain injury related to inflammation, spinal cord injury related to infection, spinal cord injury related to inflammation, brain injury related to environmental toxin, spinal cord injury related to environmental toxin, and combinations thereof.

6. The method of claim 1, wherein said neurologically related condition is selected from learning disorders, memory disorders, autism, attention deficit disorders, narcolepsy, sleep disorders, cognitive disorders, epilepsy, temporal lobe epilepsy, and combinations thereof.

7. The method of claim 3, wherein said psychiatric condition comprises depression.

8. The method of claim 7, wherein said method further comprises administering an anti-depressant agent to said subject or patient.

9. The method of claim 7, wherein said depression is due to morphine use by the subject or patient.

10. The method of claim 1, wherein said 4-acylaminopyridine derivative is 2-(2-oxypyrrolidin-1-yl)-N-(2,3-dimethyl-5,6,7,8-tetrahydrofuro(2,3-b)quinolin-4-yl)acetoamide.

11.-12. (canceled)

13. A method of preparing cells or tissue for transplantation to a subject or patient, said method comprising:
stimulating or increasing neurogenesis in said cell or tissue by contacting said cell or tissue with a 4-acylaminopyridine derivative.

14.-16. (canceled)

17. The method of claim 13, wherein said method further comprises contacting said cell or tissue with an opiod or non-opioid neurogenic agent.

18. The method of claim 17, wherein said non-opioid neurogenic agent is a muscarinic receptor ligand, such as sabcomeline.

19.-20. (canceled)

21. The method of claim 17, wherein said opioid is a kappa opioid receptor antagonist.

22.-23. (canceled)

24. The method of claim 17, wherein said cell or tissue exhibits decreased neurogenesis or is subjected to an agent which decreases or inhibits neurogenesis.

25.-27. (canceled)

28. A method of stimulating or increasing neurogenesis in a patient in need thereof, said method comprising:
administering a 4-acylaminopyridine derivative to said patient.
